(19) United States
(12) Patent Application Publication
(10) Pub. No.: US 2025/0152675 A1
(43) Pub. Date: May 15, 2025

(54) I-TYPE RECOMBINANT COLLAGEN WITH HIGH TRANSDERMAL ABSORBABILITY, AND USE THEREOF

(71) Applicant: XI'AN GIANT BIOGENE TECHNOLOLGY CO., LTD, Shaanxi (CN)

(72) Inventors: Daidi FAN, Shaanxi (CN); Weigang YUWEN, Shaanxi (CN); Zhiguang DUAN, Shaanxi (CN); Ru XU, Shaanxi (CN); Jianya YAN, Shaanxi (CN); Liping JIA, Shaanxi (CN); Chanyuan YANG, Shaanxi (CN)

(73) Assignee: XI'AN GIANT BIOGENE TECHNOLOLGY CO., LTD, Shaanxi (CN)

(21) Appl. No.: 18/833,593

(22) PCT Filed: Jan. 19, 2023
(86) PCT No.: PCT/CN2023/073219

§ 371 Date: Jul. 26, 2024

(30) Foreign Application Priority Data
Jan. 27, 2022 (CN) 202210102466.7

(51) Int. Cl. A61K 38/39; C07K 14/78

(52) U.S. Cl. CPC A61K 38/39; C07K 14/78

(57) ABSTRACT

The present invention relates to an I-type recombinant collagen with high transdermal absorbability, and the use thereof. The human I-type recombinant collagen with high transdermal absorbability of the present invention is composed of multiple repetitions of a short amino acid sequence from natural human I-type collagen as a repeating unit, wherein the short amino acid sequence is as shown in SEQ ID NO: 1, and the number of repetitions is three or more. The human I-type recombinant collagen with high transdermal absorbability of the present invention can be used as a raw material for producing various collagen products, such as an injection, a facial filler, a dressing, cosmetics, a health-care food product, tissue engineering material and a collagen sponge.

### FIELD OF THE INVENTION

The application belongs to the field of biotechnology, and in particular relates to a type I recombinant collagen with high transdermal absorbability and use thereof.

### BACKGROUND OF THE INVENTION

Collagen protein is a biological polymer protein, the main component of animal connective tissue, and the most abundant and widely distributed functional protein in mammals, accounting for 25% to 30% of the total protein. Collagen protein is closely related to the formation and maturation of tissues, the transmission of information between cells, joint lubrication, wound healing, calcification, blood coagulation and aging, etc., is one of the most critical raw materials in the biotechnology industry, and is widely used in medical materials, cosmetics and food industries.

Collagen protein, also known as collagen, is an important protein component of connective tissue that supports and protects the body, and is also the most abundant structural protein in many vertebrates and invertebrates, which provides mechanical strength to bones, tendons, cartilage and skin. Collagen is one of the most abundant proteins in mammals, accounting for about 20%-30% of the total protein in the body. It mainly exists in skin, bones, muscle tendons, soft tissues, etc., wherein, about 70%-80% of the extracellular matrix of the skin is collagen. Collagen is closely related to the formation and maturation of tissues, the transmission of information between cells, joint lubrication, wound healing, calcification, blood coagulation and aging, etc., is one of the most critical raw materials in the biotechnology industry, and is widely used in the field of medical treatment or beauty.

When collagen is used in medical dressings, cosmetics, etc., it is hoped that collagen has good transdermal absorption performance from the perspective of better biological activity. However, collagen is a macromolecular bioactive substance, which itself is not easily absorbed through the skin. Therefore, in previous studies, how to promote the transdermal absorption of collagen has become a research hotspot. For example, in order to promote the transdermal absorption of collagen molecules with a large molecular weight, a mask towel-type collagen dressing may be used, that is, the collagen solution is impregnated in a solid carrier such as non-woven fabric, then sealed in a container, taken out before use, and applied on the face. In the above-mentioned mask towel-type collagen dressing, the collagen solution is impregnated in the solid carrier with liquid absorption property, which can make more collagen solution contained and delay the collagen solution drying up, and thus the interaction time between the collagen solution and the skin surface can be prolonged and promotes the penetration of collagen molecules with large molecular weight.

In recent years, with the wide application of genetic engineering technology, researchers have created various types of recombinant collagen. For example, recombinant collagen may be constructed by selecting and repeating a short amino acid sequence derived from natural human collagen. The recombinant collagen constructed in this way has advantages such as low immunogenicity, high biological activity, and good stability. Theoretically, the transdermal absorption performance of this recombinant collagen may be related to its amino acid sequence, which has not been clearly confirmed. Especially for how to design the short amino acid sequence as a repeating unit, so that the constructed recombinant collagen has better transdermal absorption performance, there is no theory in the prior art that can be used as a guide.

### SUMMARY OF THE INVENTION

In order to solve the above-mentioned technical problems in the prior art, the inventors conducted in-depth research, and as a result obtained a short amino acid sequence derived from natural human type I collagen and a type I recombinant collagen constructed using the short amino acid sequence as a repeating unit, the type I recombinant collagen having excellent transdermal absorption performance, thereby completing the present application.

That is, the present application includes:
1. A polypeptide derived from natural human type I collagen, having an amino acid sequence as shown in SEQ ID No.: 1 (GAPGAPGSQGAPGLQGMPGERGAAGLPGPKGDRGDA).
2. An type I recombinant collagen, formed by multiple repetitions of a short amino acid sequence derived from natural human type I collagen as a repeating unit, wherein, the short amino acid sequence is shown in SEQ ID No.: 1 (GAPGAPGSQGAPGLQGMPGERGAAGLPGPKGDRGDA), and times of the repetitions is three or more.
3. The type I recombinant collagen according to item 1, wherein, the times of the repetitions is 5-150, preferably 10-100.
4. The type I recombinant collagen according to item 1, further having a tag facilitating purification thereof, the tag being a His tag, a Flag tag or a c-Myc tag.
5. Use of the type I recombinant collagen according to any one of items 1-4 in the preparation of a collagen product.
6. The use according to item 5, wherein the collagen product is selected from the group consisting of an injection, a facial filler, a dressing, a cosmetic, a health food, a tissue engineering material, a collagen sponge and the like.

### BRIEF DESCRIPTION OF THE DRAWINGS

FIG. 1 is the SDS-PAGE protein electrophoresis diagram of purified recombinant collagens P-1˜P-4.

FIG. 2 is the SDS-PAGE protein electrophoresis diagram of purified recombinant collagens D-1˜D-4.

### DETAILED DESCRIPTION OF THE INVENTION

The present application will be described in detail through specific examples below. It should be pointed out that these descriptions are only exemplary and do not limit the scope of the present application.

We firstly screened various short amino acid sequences derived from natural human type I collagen (such short amino acid sequences are 100% homologous to natural human type I collagen, which can avoid problems such as immunogenicity of foreign substances), to obtain a short amino acid sequence with excellent transdermal absorption performance. Then, type I recombinant collagen with various molecular weights is constructed using the short amino acid sequence as a repeating unit, and its transdermal absorption performance is verified. Therefore, the type I recombinant collagen with excellent transdermal absorption performance is obtained.

It should also be noted that the collagen having good adhesion-promoting effect is an important reason why it may be widely used in implanted medical devices. The implanted collagen medical devices can promote the fibroblasts, adipocytes, dermal cells, etc. to migrate to the implanted device, and exhibit cell adhesion, promoting cell growth and other characteristics, achieving rapid repair. GER tripeptide is a known tripeptide with adhesion effect, and collagen containing this tripeptide exhibits better adhesion-promoting effect. In order to ensure the physiological activity of the obtained type I recombinant collagen with excellent transdermal absorption performance, when screening the short amino acid sequence with high transdermal absorption performance, the natural short amino acid sequence containing GER tripeptide was preferentially selected, and then was repeated to construct a new type I recombinant collagen.

### Example 1: Preparation of Polypeptide With High Transdermal Absorbability

### 1) Preparation of Collagen Peptide

The telopeptide amino acid sequences at both ends of the natural amino acid sequence of type I collagen were removed, remaining 1057 amino acids (162-1219) in the middle. The fragments containing GER tripeptide were screened using amino acid analysis software, with 36 amino acids as the basic repeating unit and 18 amino acids being repeated between different short peptides, and a total of 27 polypeptide fragments were screened out. The 27 short peptides were synthesized by chemical synthesis, and 27 pure short peptides were prepared with a purity of more than 95%. The polypeptides were synthesized by Sichuan Pukang Pharmaceutical Co., Ltd. and confirmed by mass spectrometry and high performance liquid chromatography.

### 2) Comparison of Transdermal Performance

1. Preparation of Mouse Skin Ex Vivo 10 Kunming mice of 20 to 22 g with half male and half male were used in the experiment. After the mice were killed, the hairs on the abdomen were removed. Then the skin on the hair-removed area was peeled off, stripped of fat and tendons, and rinsed repeatedly with distilled water. After being washed, the mouse skins were rinsed with normal saline, then treated with 10% glycerol, and stored at −20° C. for use (use up within 7 days).

### 2. Experimental Device

Single-chamber diffusion cell: The effective diffusion area of the diffusion cell is 2.0 cm², the volume of the receiving cell is 14 ml, the length of the custom-made stirrer is 1.4 cm, and the receiving solution is 0.9% NaCl solution.

### 3. Preparation of Sample Solution

The stored mouse skin was taken out, thawed, washed with normal saline, and sandwiched between the receiving chamber and the supplying chamber, with the medicated side facing the supplying chamber and the skin side facing the receiving chamber. The temperature of the water bath system was adjusted to 37.5° C., and the stirring speed was adjusted to 100 rpm/min. 0.9% NaCl solution pre-warmed at 37° C. was added to the receiving chamber and the air bubbles were exhausted, in order to reduce the interference, the inner surface of the mouse skin was firstly contacted with the receiving solution without medication and all the receiving solution was replaced. Then the 27 collagen peptides synthesized were prepared into 5 mg/ml solutions using 0.9% NaCl respectively, which were injected into the supplying chamber and firmly adhere to the mouse skin respectively. After 24 h, part of the receiving solution was absorbed with a syringe as the sample solution to determine the transdermal amount of collagen peptide.

### 4. Detection of Target Peptide

The BCA kit method was used to determine the polypeptide content in the sample solution.

### 5. Results of Transdermal Absorption

TABLE 1
Comparison of the 24h penetration amount of different collagen peptides

| No. | Amino acid sequence | 24h penetration amount (ug) |
|---|---|---|
| 1 | gepgasgpmgprgppgppgkn ddgeagkpgrpger | 2023.41 |
| 2 | gkngddgeagkpgrpgergppgpqgarglpgtaglp | 2097.44 |
| 3 | gergppgpq garglpgtag lpgmkghrgfsgldgak | 2224.03 |
| 4 | gakgdagpagpkgepgspgengapgqmgprglpger | 2034.78 |
| 5 | gengapgqmgprglpgergrpgapgpagargndgat | 2123.45 |
| 6 | gergrpgapgpagargndgatgaagppgptgpagpp | 2233.35 |
| 7 | gpvgvqgpp gpageegkrg argepgptglpgppger | 2189.90 |
| 8 | gargepgptglpgppgerggp gsrgfpgadgvagpk | 2013.46 |
| 9 | gerggpgsrgfpgadgvagpkgpagergspgpagpk | 2341.24 |
| 10 | grpgppgppgargqagvmgfpgpkgaagepgkager | 2290.34 |
| 11 | gfpgpkgaagepgkagergvpgppgavgpagkdgea | 2576.34 |
| 12 | gergvpgppgavgpagkdgeagaqgppgpagpager | 2613.34 |
| 13 | glpgpagppgeagkpgeqgvpgdlgapgpsgarger | 2098.04 |
| 14 | gvpgdlgapgpsgargergfpgergvqgppgpagpr | 2045.62 |
| 15 | gergvqgppgpagprgangapgndgakgdagapgap | 2111.23 |
| 16 | gangapgndgakgdagapgapgsqgapglqgmpger | 2034.34 |
| 17 | gapgapgsqgapglqgmpgergaaglpgpkgdrgda | 2998.75 |
| 18 | gergaaglpgpkgdrgdagpkgadgspgkdgvrglt | 2309.24 |
| 19 | gspgadgpagapgtpgpqgiagqrgvvglpgqrger | 2432.23 |
| 20 | giagqrgvvglpgqrgergfpglpgpsgepgkqgps | 2509.11 |
| 21 | gergfpglpgpsgepgkqgpsgasgergppgpmgpp | 2199.78 |
| 22 | gvvglpgqrgergfpglpgpsgepgkqgpsgasger | 2092.23 |
| 23 | gepgkqgpsgasgergppgpmgppglagppgesgre | 2323.45 |
| 24 | gergppgpmgppglagppgesgregapgaegspgrd | 2234.67 |
| 25 | gppgeagkpgeqgvpgdlgapgpsgargergfpger | 2255.78 |
| 26 | gapgpsgargergfpgergvqgppgpagprgangap | 2054.99 |
| 27 | gergvqgppgpagprgangapgndgakgdagapgap | 2186.24 |

The results show that the polypeptide with the sequence number 17 and amino acid sequence of gapgapgsqgapglqgmpgergaaglpgpkgdrgda (SEQ ID No.: 1) has the largest 24 h transdermal amount of 2998.75 ug.

### Example 2 Preparation of Various Type I Recombinant Collagens Using E. coli Expression System

### 1) Preparation of Type I Recombinant Collagens With Different Repetition Times of SEQ ID No.: 1

The amino acid sequence of SEQ ID No.: 1 was repeated 3 times (P-1), 10 times (P-2), 20 times (P-3), 40 times (P-4) respectively, optimized by the codon preference of E. coli, and then translated into the corresponding gene sequences. The gene sequences were connected into the pET24a expression plasmids after the whole gene synthesis, and transferred into BL21 competent cells by heat shock transformation to become expression strains (four kinds in total).

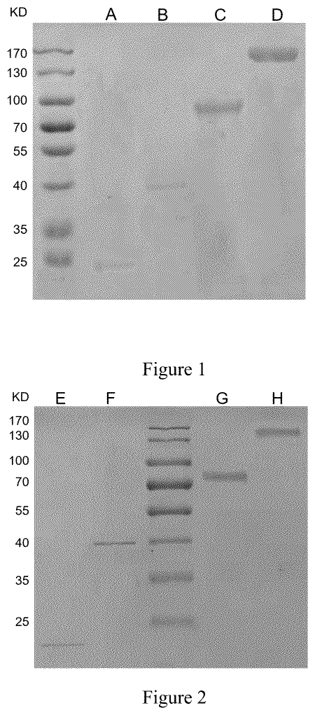

Single colonies of the above four kinds of expression strains were picked respectively, and transferred to LB liquid shake flasks to be cultured with shaking at 37° C. overnight to obtain the seed liquids. The seed liquids were transferred into 100 ml LB liquid mediums at 1% inoculation amount, cultured at 37° C. and 200 rpm, added with IPTG with a final concentration of 1.5 mM when the OD value was around 2 to 3, and cooled down to 28° C. for induction culture for 14 hours. The bacteria were collected by centrifugation, and prepared into 10% (wet weight of bacteria/PB volume) bacterial suspension with PB buffer at pH 6.0, which was homogenized under high pressure of 1000 bar for 3 min. The supernatant was collected by centrifugation to obtain the crude protein expression solution, which was purified by ion exchange chromatography. Proteins of 11.9 KD (P-1), 39.6 KD (P-2), 79.2 KD (P-3), and 158.4 KD (P-4) were collected respectively, which are type I recombinant collagens with different repetition times. The SDS-PAGE protein electrophoresis diagrams of various purified collagens are shown in FIG. 1, and lanes A, B, C, and D correspond to P-1, P-2, P-3, and P-4, respectively.

### 2) Expression of Recombinant Type I Collagen Corresponding to Other Type I Collagen Amino Acid Sequences Containing GER Tripeptide With the Same Repetition Times

The amino acid sequence of No. 12 with better transdermal absorption performance in Table 1 was repeated 4 times (D-1), 12 times (D-2), 24 times (D-3), 48 times (D-4) respectively, optimized by the codon preference of E. coli, and then translated into the corresponding gene sequences. The gene sequences were connected into the pET24a expression plasmids after the whole gene synthesis, and transferred into BL21 competent cells by heat shock transformation to become the expression strains (four kinds in total).

Single colonies of the above four expression strains were picked respectively. and transferred to LB liquid shake flasks to be cultured with shaking at 37° C. overnight to obtain seed liquids. The seed liquids were transferred into 100 ml LB liquid medium with 1% inoculation amount, and cultured at 37° C. and 200 rpm. added with IPTG with a final concentration of 1.5 mM when the OD value was around 2 to 3. and cooled down to 28° C. for induction culture for 14 hours. The bacteria were collected by centrifugation. and prepared into 10% (wet weight of bacteria/PB volume) bacterial suspensions with PB buffer at pH 6.0, which were homogenated under high pressure of 1000 bar for 3 min. The supernatant were collected by centrifugation to obtain the crude protein expression 25 solutions, which were purified by ion exchange chromatography. Proteins of 12.7 KD (D-1), 37.9 KD (D-2), 78.28 KD (D-3), and 164.95 KD (D-4) were collected respectively, which are recombinant collagens with different repetition times. The SDS-PAGE protein electrophoresis diagrams of various purified recombinant collagens are shown in FIG. 2, and lanes E, F, G, and H correspond to D-1, D-2, D-3, and D-4, respectively.

### Example 3 Comparison of Transdermal Absorption Performance of Various Type I Recombinant Collagens

For the 8 kinds of recombinant collagens prepared in Example 2, permeated protein was measured by the method in comparison of transdermal performance in Example 1. The results are shown in Table 2.

TABLE 2
Comparison of the 24 h permeation amount of the recombinant
collages with different amino acid sequences

| No. | Recombinant protein | 24 h permeation amount/ug |
|---|---|---|
| 1 | P-1 | 1523.84 |
| 2 | P-2 | 1237.93 |
| 3 | P-3 | 1024.49 |
| 4 | P-4 | 893.42 |
| 5 | D-1 | 1241.21 |
| 6 | D-2 | 1090.32 |
| 7 | D-3 | 676.23 |
| 8 | D-4 | 413.09 |

As shown in Table 2, as the molecular weight increases, the transdermal absorption efficiency of the protein gradually decreases. However, the recombinant collagens formed by repeating SEQ ID No.: 1 screened in the present application which have similar molecular weight and similar repetition times show good transdermal absorption effect.

1. A polypeptide derived from natural human type I collagen, having an amino acid sequence as shown in SEQ ID No.: 1
(GAPGAPGSQGAPGLQGMPGERGAAGLPGPKGDRGDA).

2. An type I recombinant collagen, formed by multiple repetitions of a short amino acid sequence derived from natural human type I collagen as a repeating unit,
wherein, the short amino acid sequence is shown in SEQ ID No.: 1 (GAPGAPGSQGAPGLQGMPGERGAAGLPGPKGDRGDA), and times of the repetitions is three or more.

3. The type I recombinant collagen according to claim 1, wherein, the times of the repetitions is 5-150, preferably 10-100.

4. The type I recombinant collagen according to claim 1, further having a tag facilitating purification thereof, the tag being a His tag, a Flag tag or a c-Myc tag.

5. Use of the type I recombinant collagen according to claim 1 in the preparation of a collagen product.

6. The use according to claim 5, wherein the collagen product is selected from the group consisting of a collagen injection, a facial filler, a dressing, a cosmetic, a health food, a tissue engineering material and a collagen sponge.


SEQUENCE LISTING (1 sequence)

SEQ ID NO: 1 - protein, 36 aa, Homo sapiens
GAPGAPGSQGAPGLQGMPGERGAAGLPGPKGDRGDA